(19) United States
(12) Patent Application Publication
(10) Pub. No.: US 2010/0274205 A1
(43) Pub. Date: Oct. 28, 2010

(54) WOUND CARE KIT

(76) Inventors: Rocco R. Morelli, Lincolnshire, IL (US); Scott D. Hippensteel, Gurnee, IL (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/760,103

(22) Filed: Apr. 14, 2010

Related U.S. Application Data
(60) Provisional application No. 61/172,502, filed on Apr. 24, 2009.

(51) Int. Cl. A61B 19/02; A61L 15/44; A61M 1/00

(52) U.S. Cl. 604/290; 206/570; 604/319

(57) ABSTRACT

A wound treatment kit for use in negative pressure wound therapy comprising a first container, a second container, and a third container. The first container comprises wound and skin preparation materials, the second container comprises wound dressing materials, and the third container comprises wound sealing materials. The first container further comprises a first label providing information directing the user to use the wound and skin preparation materials first in sequence before the wound dressing materials and the wound sealing materials. The second container further comprises a second label providing information directing the user to use the wound dressing materials second in sequence after the wound and skin preparation materials and before the wound sealing materials. The third container further comprises a third label providing information directing the user to use the wound sealing materials third in sequence after the wound and skin preparation materials and the wound dressing materials.

### CROSS-REFERENCE TO RELATED APPLICATION

This application claims the priority benefit of U.S. Provisional Application No. 61/172,502 filed on Apr. 24, 2009, the entire disclosure of which is incorporated herein by reference.

### BACKGROUND

The present invention generally relates to a kit containing multiple containers or chambers supplying equipment for use in wound care, including negative pressure wound treatment (sometimes called negative pressure wound therapy), and labels instructing the user of the order in which to access the containers/chambers.

### SUMMARY OF THE INVENTION

Described herein is a wound treatment kit. The wound treatment kit can comprise a first container, a second container, and a third container. The first container can comprise wound and skin preparation materials. The second container can comprise wound dressing materials. The third container can comprise wound sealing materials.

The first container can further comprise a first label that provides information directing the user to use the wound and skin preparation materials first in sequence before the wound dressing materials and the wound sealing materials. The second container can further comprise a second label that provides information directing the user to use the wound dressing materials second in sequence after the wound and skin preparation materials and before the wound sealing materials. The third container can further comprise a third label that provides information directing the user to use the wound sealing materials third in sequence after the wound and skin preparation materials and the wound dressing materials.

The wound and skin preparation materials can comprise a barrier wipe and a ruler. The wound dressing materials can comprise a drain, a non-adherent dressing, scissors, and dressing means to eliminate dead space between the wound bed and film dressing. The wound sealing materials can include a film dressing and paste. The dressing means to eliminate dead space can include gauze sponge, foam, synthetic gauzes and meshes, and non-synthetic dressings such as cotton gauze that are sized to a volume appropriate to fill dead space between the wound and the film dressing when negative pressure wound treatment is occurring.

The containers can take the form of trays. For example, the first container can comprise a first tray, the second container can comprise a second tray, and the third container can comprise a third tray. The first tray can comprise a first open top and a first wrapper. The second tray can comprise a second open top and a second wrapper. The first wrapper can at least partially close the first open top. The second wrapper can at least partially close the second open top. The third container can at least partially surround the first container and second container. For example, the first container and second container can be sized so as to fit within the space of the third container. A wrapper can enclose the first container, the second container, and the third container.

Alternatively, the containers can take the form of chambers formed within in a contiguous structure. For example, the kit can comprise a contiguous structure. The contiguous structure can comprise a first chamber, a second chamber, and a third chamber. The first chamber can comprise the wound and skin preparation materials. The second chamber can comprise the wound dressing materials. The third chamber can comprise the wound sealing materials. The contiguous structure can comprise a first perforation between the first chamber and the second chamber and a second perforation between the second chamber and the third chamber. The perforations would allow the chambers to be separated from one another.

Like the first container, the first chamber can comprise a first label providing information directing the user to use the wound and skin preparation materials first in sequence before the wound dressing materials and the wound sealing materials. Like the second container, the second chamber can further comprise a second label providing information directing the user to use the wound dressing materials second in sequence after the wound and skin preparation materials and before the wound sealing materials. Like the third container, the third chamber can further comprise a third label providing information directing the user to use the wound sealing materials third in sequence after the wound and skin preparation materials and the wound dressing materials.

The contiguous structure can comprise a structure width. The first chamber can comprise a first width. The second chamber can comprise a second width. The third chamber can comprise a third width. In one embodiment, the third width can be both approximately equal to the structure width and greater than or equal to the combination of the first width and the second width.

Additionally described herein is a method of preparing, dressing, and sealing a wound for treatment, such as negative pressure wound treatment, comprising the steps of (in no stringent order) (a) obtaining an embodiment of the wound treatment kit described above, (b) removing the barrier wipe from the first container or first chamber, (c) presenting a patient, the patient comprising a wound and healthy skin around the wound, (d) wiping the healthy skin with the barrier wipe, (e) removing the non-adherent dressing from the second container or second chamber, (f) placing the non-adherent dressing over the wound, (g) removing the drain from the second container, (h) placing the first end of the drain over the non-adherent dressing, (i) removing the film dressing from the third container or third chamber, (j) placing the film dressing over the wound, non-adherent dressing, and the first end of the drain, allowing the second end of the drain to not be covered by the film dressing, and (k) connecting the second end of the drain to a source of suction.

### BRIEF DESCRIPTION OF THE SEVERAL VIEWS OF THE DRAWINGS

FIG. 1 depicts a front, partially exploded, perspective view of a first embodiment of the kit with contained objects;

FIG. 2 depicts a front perspective view of the first embodiment of the kit as contained within a wrapper;

FIG. 3 depicts an overhead view of a second embodiment of the kit;

FIG. 4 depicts a perspective view of a third embodiment of the kit; and

FIG. 5 depicts an exploded perspective view of fourth embodiment of the kit.

### DETAILED DESCRIPTION OF THE PREFERRED EMBODIMENTS

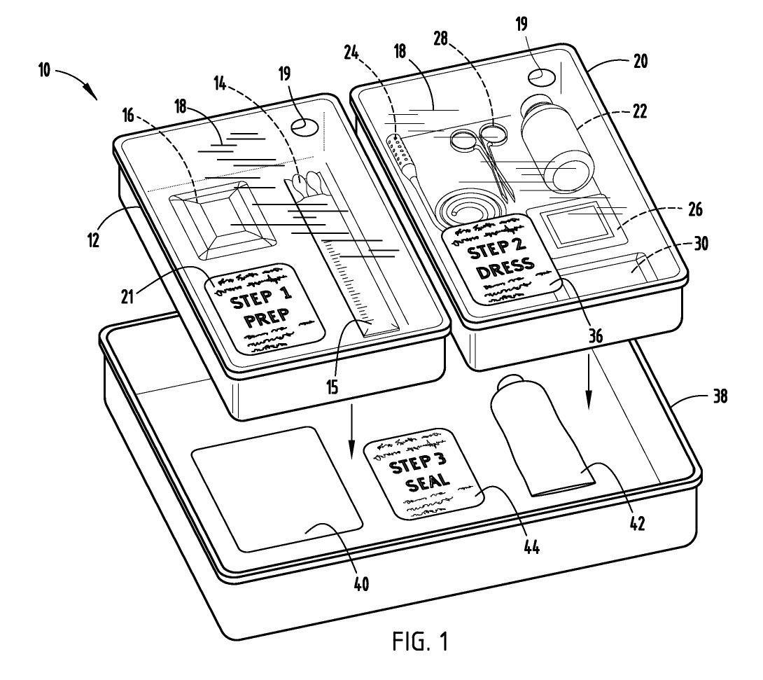

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations and step sequences, except where expressly specified to the contrary. It is also to be understood that the specific devices and processes illustrated in the attached drawings, and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

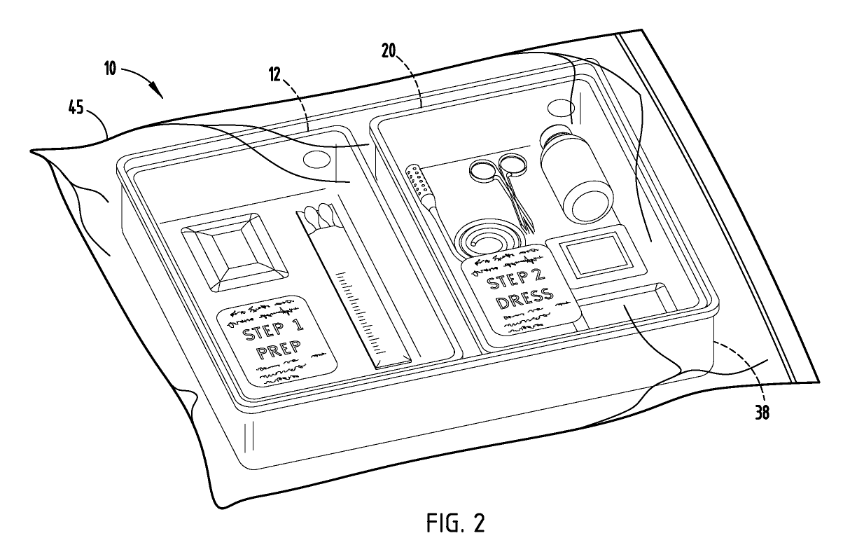

The FIGS. 1 and 2 depict a first embodiment of wound treatment kit 10. The wound treatment kit 10 can comprise a first container 12, a second container 20, and a third container 38. The first container 12 can comprise wound and skin preparation materials. The second container 20 can comprise wound dressing materials. The third container 38 can comprise wound sealing materials. “Wound and skin preparation materials” means items that are useful to prepare a wound and surrounding tissue for treatment of the wound, such as with negative pressure wound treatment. “Wound dressing materials” means items that are useful to dress a wound and, possibly, the surrounding skin for treatment of the wound, such as with negative pressure wound treatment. “Wound sealing materials” means items that a user may find useful to seal a wound and, possibly, the surrounding skin for treatment of the wound, such as with negative pressure wound treatment.

The first container 12 can further comprise a first label 21 that provides information directing the user to use the wound and skin preparation materials first in sequence before the wound dressing materials and the wound sealing materials. For example, the first label 21 can state “STEP 1 PREP” or “A PREP” (or some other statement that the contents of the first container 12 should be used first), provide directions for use, provide warnings, and have appropriate trademarks. The second container 20 can further comprise a second label 36 that provides information directing the user to use the wound dressing materials second in sequence after the wound and skin preparation materials and before the wound sealing materials. For example, the second label 36 can state “STEP 2 DRESS” or “B DRESS” (or some other statement that the contents of the second container 20 should be used second in sequence), provide directions for use, provide warnings, and have appropriate trademarks. The third container 38 can further comprise a third label 44 that provides information directing the user to use the wound sealing materials third in sequence after the wound and skin preparation materials and the wound dressing materials. For example, the third label 44 can state “STEP 3 SEAL” or “C SEAL” (or some other statement that the contents of the third container 38 should be used third in sequence), provide directions for use, provide warnings, and have appropriate trademarks.

The wound and skin preparation materials can comprise an applicator 14, a protective barrier wipe 16, and a ruler 15. The applicator 14 can be item number MDS202000 manufactured by Medline Industries, Inc. (Mundelein, Ill.), which is a cotton tipped applicator. The package for the applicator 14 can include the ruler 15, or the ruler 15 can be a standalone ruler 15. The protective barrier wipe 16 can be item number MSC1505, Sureprep® No-Sting, manufactured by Medline Industries, Inc. (Mundelein, Ill.). Other wound and skin preparation materials include wound cleansers, such as hydrogen peroxide and saline.

The wound dressing materials can comprise sterile saline solution 22, a drain 24, a non-adherent dressing 26, scissors 28, and dressing means 30 to eliminate dead space between the wound bed and film dressing 40 when in use. The dressing means 30 to eliminate dead space can include gauze sponge, foam, synthetic gauzes and meshes, and non-synthetic dressings such as cotton gauze that are sized to a volume appropriate to fill dead space between the wound and the film dressing when negative pressure wound treatment is occurring. For example, the dressing means 30 can be an appropriately sized item no. 6662, Kendall Kerlix™ AMD™, which is an antimicrobial super sponge distributed by Tyco Healthcare Group LP (Mansfield, Mass.). The sterile saline solution 22 can be item number 1022, Kendall Curity™, distributed by Tyco Healthcare Group LP (Mansfield, Mass.) or item number 3001-00, Sodium Chloride 0.9% w/v, Saljet, distributed by Winchester Laboratories (Saint Charles, Ill.). The drain 24 can be item no. 370007, Surgidyne®, manufactured by Aspen Surgical (Caledonia, Mich.), which is a perforated silicone drain, a Medline® silicone perforated drain, or Degania silicone perforated drain. The drain 24 can take other forms such as a simple tube, a multi-lumen tube, a flat tube, or a round tube, and the end of the tube can be a pad to be placed in the wound area to distribute suction force to a wider area of the wound bed. The drain 24 comprises a first end, which is the end to be placed nearest the wound, and a second end, which is the end to be placed nearest to the source of suction. The non-adherent dressing 26 can be item no. ISG20D038, Invacare® Oil Emulsion dressing, distributed by Invacare Supply Group (Holliston, Mass.), item no. 265258, CVS® pharmacy Non-Stick Dressing, distributed by CVS Pharmacy, Inc (Woonsocket, R.I.), or Medline® oil emulsion non-adherent. The non-adherent dressing 26 can take other forms such as petrolatum soaked cotton gauze and polymer meshes. The non-adherent dressings can have healing agents such as a silver-ion emitter. The scissors 28 can be angled scissors. The wound dressing materials can contain other materials useful for dressing the wound, such as antimicrobial, antiseptic, or antibiotic solutions, creams, ointments, sprays, powders (such as silver-ion powder), and gels.

The wound sealing materials can include a film dressing 40 and paste 42. The film dressing 40 can be item nos. 9536HP, 1628, or 1629, Tegaderm™ HP, manufactured by 3M Health Care (St. Paul, Minn.), which is a transparent film dressing. The film dressing 40 can be essentially impermeable, such as polyethylene, or semi-permeable, meaning essentially impermeable to liquid but permeable to a certain degree to gases (such as water-vapor and/or oxygen). The film dressing 40 can be a hydrocolloid or a hydrogel. The paste 42 can be Coloplast ostomy paste. The wound sealing materials can include tape, such as Hy-Tape by HyTape International, and hydrochloride gel tape.

The containers 12, 20, and 38 can take the form of trays. For example, the first container 12 can comprise a first tray 12 the second container 20 can comprise a second tray 20, and the third container 38 can comprise a third tray 38, as shown in FIGS. 1 and 2. The first tray 12 can comprise a first open top and a first wrapper 18. The second tray 20 can comprise a second open top and a second wrapper 18. The first wrapper 18 can at least partially close the first open top. The second wrapper 18 (the wrapper 18 over second tray 20) can at least partially close the second open top. The third container 38 can at least partially surround the first container 12 and second container 20. For example, the first container 12 and second container 20 can be sized so as to fit within the space of the third container 38. As shown in FIG. 2, a wrapper 45 can enclose the first container 12, the second container 20, and the third container 38. The wrapper 45 can be a resealable bag.

The wrappers 18 and 45 can be an at least partially transparent plastic film. The plastic film can be a bioplastic, a plastic derived from renewable biomass sources, such as wood (wood cellulose) vegetable oil, cornstarch, pea starch, microbiota, and cane sugar (including polylactide acid), among others. The wrappers 18 and 45 can take other forms, such as paper wrappers and cardboard boxes. As shown in FIG. 1, wrappers 18 can incorporate a hole 19 in the wrapper 18 to allow the insertion of a finger to allow the user to open the wrapper 18 easily to access the contents of the respective container 12, 20. Instead of a hole 19, the wrapper 18 can incorporate a flap or a zipper.

Instead of open top trays, other forms for one or more of the containers 12, 20, and 38 are envisioned, such as fully enclosed boxes. The containers 12, 20, and 38 can be made from a bioplastic, as well. The kit 10 can contain other instructions for use.

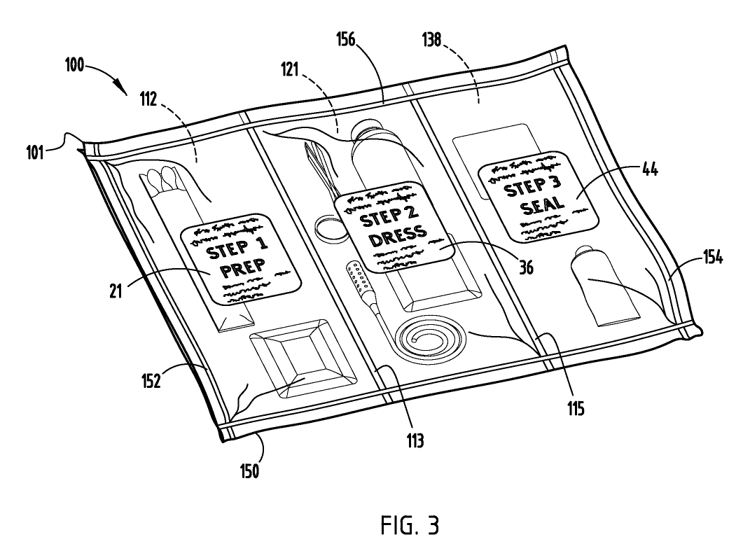

Instead of containers, as shown in FIGS. 3-5, the kit can comprise a contiguous structure with chambers formed in the contiguous structure. The contiguous structure can comprise a first chamber, a second chamber, and a third chamber. The first chamber can comprise the wound and skin preparation materials, discussed above. The second chamber can comprise the wound dressing materials, discussed above. The third chamber can comprise the wound sealing materials, discussed above.

Like the first container 12, the first chamber can comprise the first label 21, discussed above, providing information directing the user to use the wound and skin preparation materials first in sequence before the wound dressing materials and the wound sealing materials. Like the second container 21, the second chamber can further comprise the second label 36, discussed above, providing information directing the user to use the wound dressing materials second in sequence after the wound and skin preparation materials and before the wound sealing materials. Like the third container 38, the third chamber can further comprise the third label 44, discussed above, providing information directing the user to use the wound sealing materials third in sequence after the wound and skin preparation materials and the wound dressing materials.

As an example embodiment, FIG. 3 shows kit 100, which comprises the contiguous structure of bag 101. Bag 101 can comprise first chamber 112, second chamber 121, and third chamber 138. Bag 101 can comprise seals 113 and 115 and those seals 113 and 115 can divide the three chambers 112, 121, and 138 in bag 101. Bag 101 can be formed from a single sheet of plastic, which can be a bioplastic, as described above. The single sheet of plastic can be folded in half, with the fold 150 forming the bottom of the inside of the bag 101. The sides of the bag 101 can be formed by sealing the folds of the sheet of plastic together at seals 152 and 154. Seals 113 and 115 are then added to the sheet of plastic to form partially the three chambers 112, 121, and 138. The wound and skin preparation materials, described above, can be added to first chamber 112. The wound dressing materials, described above, can be added to second chamber 121. The wound sealing materials, described above, can be added to third chamber 138. The chambers 112, 121, and 138 can then be fully made by sealing the top of the bag 101 at seal 156. Seals 152, 154, 113, 115, and 156 can be a heat seal, an adhesive seal, or a zip-lock type seal. Bag 101 can further comprise first label 21, described above, adjacent to first chamber 112. Bag 101 can further comprise second label 36, described above, adjacent to second chamber 121. Bag 101 can further comprise third label 44, described above, adjacent to third chamber 138.

Bag 101 can comprise a first perforation at seal 113 between the first chamber 112 and the second chamber 121 and a second perforation at seal 115 between the second chamber 121 and the third chamber 138. The perforations allow the chambers 112, 121, and 138 to be separated from one another. Kit 100 can include further instructions for use.

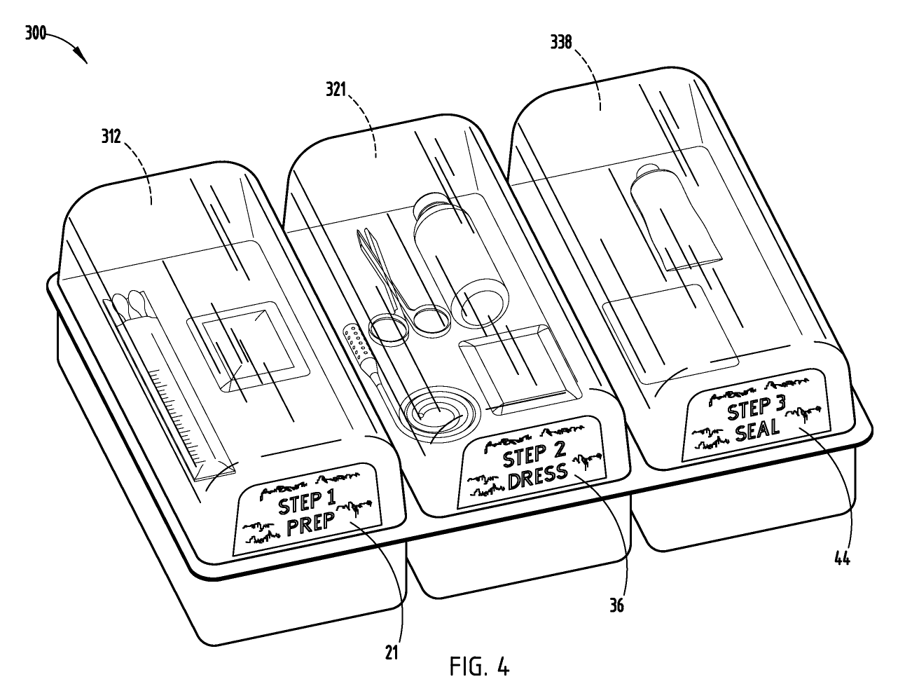

Another embodiment is kit 300 (FIG. 4), which is a contiguous structure that can be made from rigid molded plastic and can be of the clam-shell variety. The kit 300 comprises first chamber 312, second chamber 321, and third chamber 338. First chamber 312 can hold the wound and skin preparation materials, described above. Second chamber 321 can hold the wound dressing materials, described above. Third chamber 338 can hold the wound sealing materials, described above. Kit 300 can further comprise first label 21, described above, adjacent to first chamber 312. Kit 300 can further comprise second label 36, described above, adjacent to second chamber 321. Kit 300 can further comprise third label 44, described above, adjacent to third chamber 338. Kit 300 can comprise a first perforation in the contiguous material between the first chamber 312 and the second chamber 321, and a second perforation in the contiguous material between the second chamber 312 and the third chamber 338. The perforations allow the chambers 312, 321, and 338 to be separated from one another. Kit 300 can include further instructions for use.

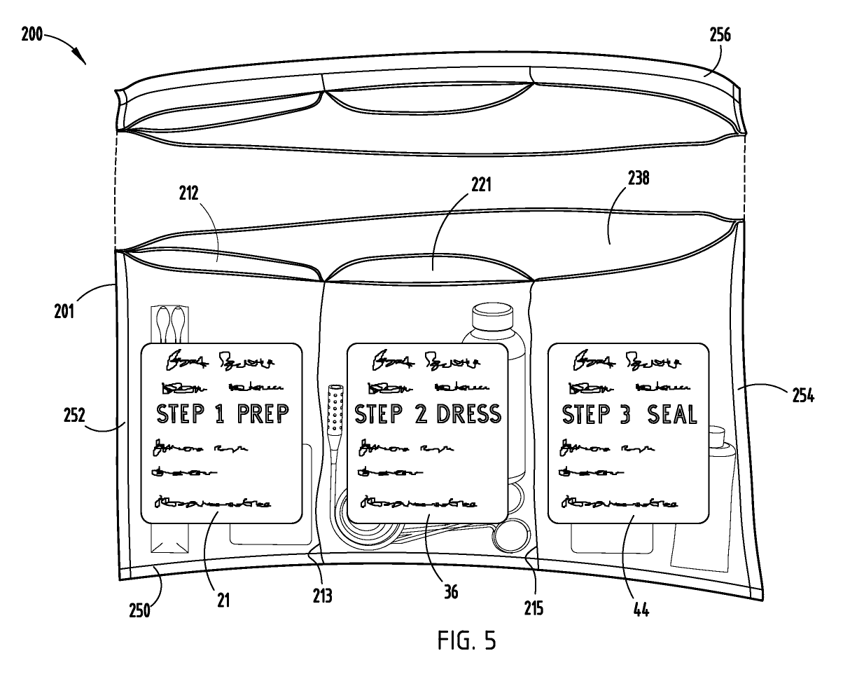

Yet another embodiment is kit 200 (FIG. 5), which comprises the contiguous structure of bag 201. Bag 201 can comprise first chamber 212, second chamber 221, and third chamber 238. Bag 201 can be formed from several sheets of plastic. The sheets of plastic can sealed at seal 250 to form the bottom of the bag 201. The sides of the bag 201 can be formed by heat sealing the appropriate sheets of plastic together at seals 252 and 254. Seals 213 and 215 are then added to the sheets of plastic to partially form the first chamber 212, the second chamber 221, and the third chamber 238, accordingly. The wound and skin preparation materials, described above, can be added to first chamber 212. The wound dressing materials, described above, can be added to second chamber 221. The wound sealing materials, described above, can be added to third chamber 238. The chambers 212, 221, and 238 can then be fully made by sealing the sheets at seal 256 to form the top of the bag 201. Seals 256, 252, 213, 215, 254, and 250 can be a heat seal, an adhesive seal, or a zip-lock type seal. Bag 201 can further comprise first label 21, described above, adjacent to first chamber 212. Bag 201 can further comprise second label 36, described above, adjacent to second chamber 221. Bag 201 can further comprise third label 44, described above, adjacent to third chamber 238.

In this embodiment, third chamber 238 is larger than first chamber 212 and second chamber 221, to accommodate a larger sized film dressing 40, for larger sized wounds, if desired. In other words, the contiguous structure of bag 201 can comprise a structure width, which is the width between seal 252 and seal 254. The first chamber 212 can comprise a first width, which is the width between seal 252 and 213. The second chamber 221 can comprise a second width, which is the width between seal 213 and seal 215. The third chamber 238 can comprise a third width, which is the width between seal 252 and seal 254. Thus, in this embodiment, the third width can be approximately equal to the structure width, and the third width can be greater than or equal to the combination of the first width and the second width. Kit 200 can include further instructions for use. The third chamber 238 can be utilized as a re-sealable chamber to collect waste generated from use of the kit.

As a method of method of preparing, dressing, and sealing a wound for treatment, such as negative pressure wound treatment, using the embodiments described herein, kit 10 will be the subject of an example. The method comprises one or more of the following steps of (in no stringent order): presenting a patient, the patient comprising a wound and healthy skin around the wound, obtaining an embodiment of the wound treatment kit described above, such as kit 10, removing the items contained in wrapper 45, obtaining the first container 12 (marked with the first label 21), placing a finger in hole 19 of wrapper 18 and removing or tearing a portion of wrapper 18, removing applicator 14 from its packaging and uses the applicator 14 to remove exudate and other debris from the patient's wound, measuring the wound size with ruler 15 if desired, removing the barrier wipe 16 from the first container 12 and wiping the patient's skin around the wound with the barrier wipe 16 (thus having prepared the wound and surrounding tissue for treatment of the wound), obtaining the second container 20 (marked with second label 36), placing a finger in hole 19 of wrapper 18 and removing or tearing a portion of wrapper 18, removing the non-adherent dressing 26 from the second container 20, deciding what dimensions the non-adherent dressing 26 should be, based on the measured dimensions of the patient's wound, obtaining the scissors 28, cutting the non-adherent dressing 26 to the decided dimensions, placing the non-adherent dressing over the wound, such as within the wound bed, obtaining the drain 24 from the second container 20, deciding what the appropriate size of the drain 24 should be, based on the measured dimensions of the patient's wound, using the scissors 28 to cut the drain 24 to the decided size, placing a first end of the drain 24 over the non-adherent dressing 26 over the patient's wound, obtaining the dressing means 30 from the first container 20, which in this example can be a sponge gauze, obtaining the saline solution 22 from the first container 20, soaking the sponge gauze in the saline solution 22, placing the saline-soaked sponge gauze 30 dressing over the non-adherent dressing 26 and drain 24 (thus having prepared the a wound and surrounding skin for treatment of the wound), obtaining the third container 38 (marked with third label 44), removing the paste 42 from the third container 38, placing a portion of the paste 42 around or under the tube 24 where the tube 24, the patient's skin, and the edge of the film dressing 40 will meet, removing the film dressing 40 from the third container 38, cutting the film dressing 40 to the proper size for the measured wound, placing the film dressing 40 over the wound, non-adherent dressing 26, and the first end of the drain 24, sponge gauze 30, allowing the second end of the drain to not be covered by the film dressing 40, making sure that the tube 24, paste 42, and film dressing 40 form an essentially air-tight seal over the skin around the wound (thus having sealed the wound for treatment), and connecting the second end of the drain 24 to a source of suction, such as a vacuum unit or a wall suction connection in a hospital room. Negative pressure wound treatment can thus commence. The other kits described herein can be used instead, with the appropriate chambers taking the place of the containers mentioned in the above method.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

What is claimed is:

1. A wound treatment kit comprising:
a first container, a second container, and a third container;
the first container comprising wound and skin preparation materials;
the second container comprising wound dressing materials; and
the third container comprising wound sealing materials.

2. The wound treatment kit of claim 1,
the first container further comprising a first label comprising information directing the user to use the wound and skin preparation materials first in sequence before the wound dressing materials and the wound sealing materials;
the second container further comprising a second label comprising information directing the user to use the wound dressing materials second in sequence after the wound and skin preparation materials and before the wound sealing materials; and
the third container further comprising a third label comprising information directing the user to use the wound sealing materials third in sequence after the wound and skin preparation materials and the wound dressing materials.

3. The wound treatment kit of claim 1, the wound and skin preparation materials comprising a barrier wipe.

4. The wound treatment kit of claim 3, the wound and skin preparation materials further comprising a ruler.

5. The wound treatment kit of claim 1, the wound dressing materials comprising a drain and a non-adherent dressing.

6. The wound treatment kit of claim 5, the wound dressing materials further comprising a scissors.

7. The wound treatment kit of claim 5, the wound dressing materials further comprising dressing means to eliminate dead space between the wound bed and film dressing.

8. The wound treatment kit of claim 1, the wound sealing materials comprising a film dressing.

9. The wound treatment kit of claim 8, the wound sealing materials further comprising a paste.

10. The wound treatment kit of claim 1, the first container comprising a first tray, the second container comprising a second tray, and the third container comprising a third tray.

11. The wound treatment kit of claim 10,
the first tray comprising a first open top and a first wrapper,
the second tray comprising a second open top and a second wrapper,
wherein the first wrapper at least partially closes the first open top, and
wherein the second wrapper at least partially closes the second open top.

12. The wound treatment kit of claim 11, the third container at least partially surrounding the first container and second container.

13. The wound treatment kit of claim 12, further comprising a wrapper enclosing the first container, the second container, and the third container.

14. A wound treatment kit comprising a contiguous structure comprising:
a first chamber, a second chamber, and a third chamber;
the first chamber comprising wound and skin preparation materials;
the second chamber comprising wound dressing materials; and
the third chamber comprising wound sealing materials.

15. The wound treatment kit of claim 14, the contiguous structure comprising a first perforation between the first chamber and the second chamber and a second perforation between the second chamber and the third chamber.

16. The wound treatment kit of claim 14,
the first chamber further comprising a first label comprising information directing the user to use the wound and skin preparation materials first in sequence before the wound dressing materials and the wound sealing materials;
the second chamber further comprising a second label comprising information directing the user to use the wound dressing materials second in sequence after the wound and skin preparation materials and before the wound sealing materials; and
the third chamber further comprising a third label comprising information directing the user to use the wound sealing materials third in sequence after the wound and skin preparation materials and the wound dressing materials.

17. The wound treatment kit of claim 16,
the wound and skin preparation materials comprising a barrier wipe,
the wound dressing materials comprising a drain and a non-adherent dressing, and
the wound sealing materials comprising a film dressing.

18. The wound treatment kit of claim 17,
the wound and skin preparation materials further comprising a ruler,
the wound dressing materials further comprising a scissors and means to eliminate dead space between the wound bed and film dressing, and
the wound sealing materials further comprising a paste.

19. The wound treatment kit of claim 16, the contiguous structure further comprising a structure width, the first chamber comprising a first width, the second chamber comprising a second width, and the third chamber comprising a third width, wherein the third width is approximately equal to the structure width, and the third width is greater than or equal to the combination of the first width and the second width.

20. A method of preparing, dressing, and sealing a wound for treatment comprising:
obtaining a wound treatment kit, the wound treatment kit comprising a first container, a second container, and a third container;
the first container comprising wound and skin preparation materials and;
the second container comprising wound dressing materials; and
the third container comprising wound sealing materials,
wherein the first container further comprises a first label comprising information directing the user to use the wound and skin preparation materials first in sequence before the wound dressing materials and the wound sealing materials;
the second container further comprising a second label comprising information directing the user to use the wound dressing materials second in sequence after the wound and skin preparation materials and before the wound sealing materials;
the third container further comprising a third label comprising information directing the user to use the wound sealing materials third in sequence after the wound and skin preparation materials and the wound dressing materials;
the wound and skin preparation materials comprising a barrier wipe;
the wound dressing materials comprising a non-adherent dressing and a drain, the drain comprising a first end and a second end;
the wound sealing materials comprising a film dressing;
removing the barrier wipe from the first container;
presenting a patient, the patient comprising a wound and healthy skin around the wound;
wiping the healthy skin with the barrier wipe;
removing the non-adherent dressing from the second container;
placing the non-adherent dressing over the wound;
removing the drain from the second container;
placing the first end of the drain over the non-adherent dressing;
removing the film dressing from the third container;
placing the film dressing over the wound, non-adherent dressing, and the first end of the drain, allowing the second end of the drain to not be covered by the film dressing; and
connecting the second end of the drain to a source of suction.
